(19) United States
(12) Patent Application Publication
(10) Pub. No.: US 2018/0057845 A1
(43) Pub. Date: Mar. 1, 2018

(54) METHOD FOR PREPARING p-VINYL PHENOLS

(71) Applicant: Universitaet Graz, Graz (AT)

(72) Inventors: Wolfgang KROUTIL, Graz (AT); Eduardo BUSTO, Oviedo (ES); Robert SIMON, Graz (AT)

(21) Appl. No.: 15/556,742

(22) PCT Filed: Mar. 3, 2016
(86) PCT No.: PCT/AT2016/050051

§ 371 Date: Sep. 8, 2017

(30) Foreign Application Priority Data
Mar. 10, 2015 (AT) A 130/2015

(51) Int. Cl. C12P 7/22; C07C 39/20; C12P 7/42; C12P 13/22

(52) U.S. Cl. CPC C12P 7/22; C12P 13/22; C12P 7/42; C07C 39/20

(57) ABSTRACT

A biocatalytic method is provided for preparing p-vinyl phenols by a three-step, one-pot reaction according to the following reaction scheme:

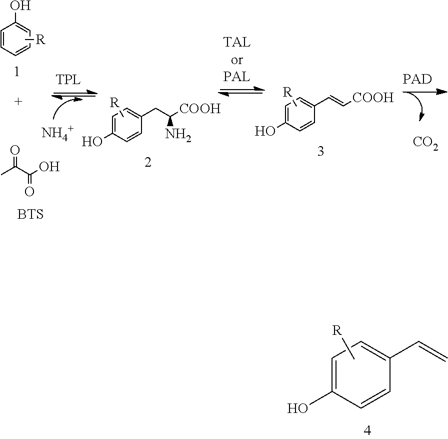

wherein the three steps include: (a) optionally substituted phenol (1) is bound to pyruvic acid (BTS) to form optionally substituted tyrosine (2) by the catalytic action of a tyrosine phenol-lyase (TPL) and in the presence of ammonium ions, (b) ammonia is eliminated from tyrosine (2) by the catalytic action of a tyrosine ammonia-lyase (TAL) or a phenyl ammonia-lyase (PAL) to produce optionally substituted p-coumaric acid (3), and (c) p-coumaric acid (3) is subjected to a decarboxylation reaction by the catalytic action of a phenolic acid decarboxylase (PAD), to produce the desired, optionally substituted p-vinyl phenol (4); and (d) wherein the generated CO₂ is removed from the reaction system to shift the chemical equilibrium of all three reaction steps (a), (b) and (c) towards the product side.

The invention relates to a biocatalytic method for preparing p-vinyl phenols.

### PRIOR ART

Vinyl phenol derivatives serve as useful components in polymer chemistry and may be used, for example, for forming dielectric layers in the production of chemical and biological sensors. Halogenated derivatives are used, for example, for producing flame retardants as well as chalcones, a well-known class of organic compounds having a wide range of biological activities.

A selective para-vinylation of non-activated phenols to para- or 4-vinyl phenols is not known. Thus direct vinylation of non-activated arenes using tin catalysts results in selective ortho-derivatization (Yamaguchi et al., J. Am. Chem. Soc. 117, 1151-1152 (1995)), whereas catalysis using Lewis acids such as GaCl₃ yields a mixture of the ortho- and para-regioisomers (Yamaguchi et al., Angew. Chem. Int. Ed. 36, 1313-1315 (1997)). However, both strategies were not performed on phenol derivatives. In fact, p-vinyl phenols were synthesized by means of Stille coupling using corresponding p-bromophenols and highly toxic vinyl-tin reagents (Littke et al., J. Am. Chem. Soc. 124, 6343-6348 (2002)).

Alternatively, p-vinyl phenols may be produced from the corresponding 4-hydroxy-benzaldehydes by means of the Heck reaction using phosphonium salt catalysis (Chen et al., Tetrahedron 69, 653-657 (2013)) or the Doebner modification of the Knoevenagel condensation by means of catalysis using secondary amines under microwave irradiation (Sinha et al., Tetrahedron 63, 960-965 (2007)).

By contrast, biocatalytic approaches are limited to the decarboxylation of p-coumaric acids (4-hydroxycinnamic acids) using ionic liquids (Sharma et al., Adv. Synth. Catal. 350, 2910-2920 (2008)), phenolic acid decarboxylases (PAD) (Wuensch et al., Org. Lett. 14, 1974-1977 (2012)), or specific p-hydroxycinnamic acid or p-coumaric acid decarboxylases, respectively (pHCA-DC or pCA-DC or, in short, PDC), such as a PDC from L. plantarum (Rodriguez et al., Proteins 78, 1662-1676 (2010) and Jung et al., Appl. Microbiol. Biotechnol. 97, 1501 (2013)), optionally in a two-phase system (Ben-Bassat et al., Org. Process Res. Dev. 11, 278-285 (2007)):

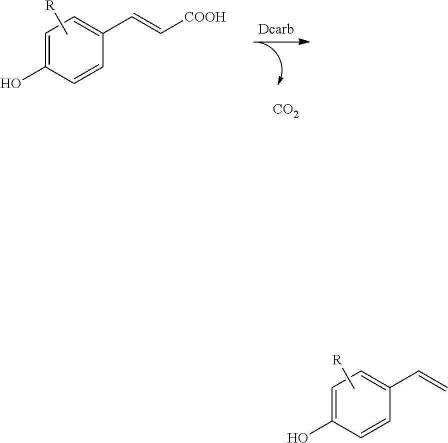

To this end, however, the corresponding cinnamic acid must be synthesized from the respective aldehyde, which is costly. Likewise, bacterial production of p-vinyl phenols from glucose using genetically engineered bacteria, which coexpress a fungal phenyl ammonia-lyase (PAL) and a bacterial p-hydroxycinnamic acid decarboxylase (PDC), by means of elimination of ammonia from tyrosine has been reported. However, pro-ductivities of this process are low (0.4 g/L), the reaction is limited to this substrate, and the amounts of by-products such as phenylalanine (0.5 g/L) and cinnamic acid are considerable (Qi et al., Metabolic Engin. 9, 268-276 (2007)).

Further the biocatalyzed synthesis of tyrosine derivatives, based on phenols, is known:

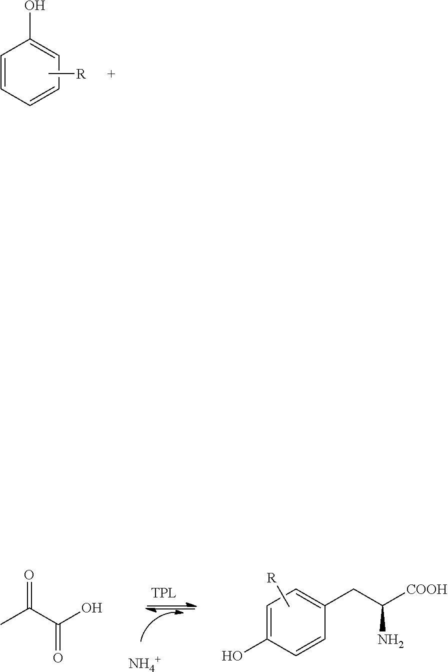

Kroutil et al., Adv. Synth. Catal. 352, 731-736 (2010), a publication by the inventors' research group, discloses this reaction for a range of phenol derivatives, including the substituents F, Cl, Br, CH₃, and OCH₃ at position 2 or 3 of the phenol, and using a tyrosine phenol-lyase (TPL) of Citrobacter freundii.

Likewise, the enzymatically catalyzed elimination of ammonia from tyrosine by means of tyrosine ammonia-lyases (TAL) or phenyl ammonia-lyases (PAL) for obtaining cinnamic acid is known:

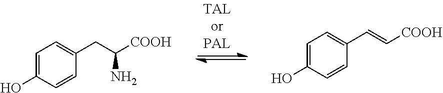 [SMILES: N[C@@H](Cc1ccc(O)cc1)C(=O)O ; O=C(O)/C=C/c1ccc(O)cc1]

See Kyndt et al., FEBS Letters 512, 240 (2002), as well as Louie et al., Chem. Biol. 13(12), 1327-1338 (2006). However, this reaction has not been reported for tyrosine so far.

In view of the above, the aim of the invention was to develop an improved biocatalytic method for producing p-vinyl phenols which enables fast and inexpensive production thereof.

### DISCLOSURE OF THE INVENTION

The invention achieves this aim by providing a biocatalytic method for producing p-vinyl phenols, comprising a three-step one-pot reaction according to the following reaction scheme:

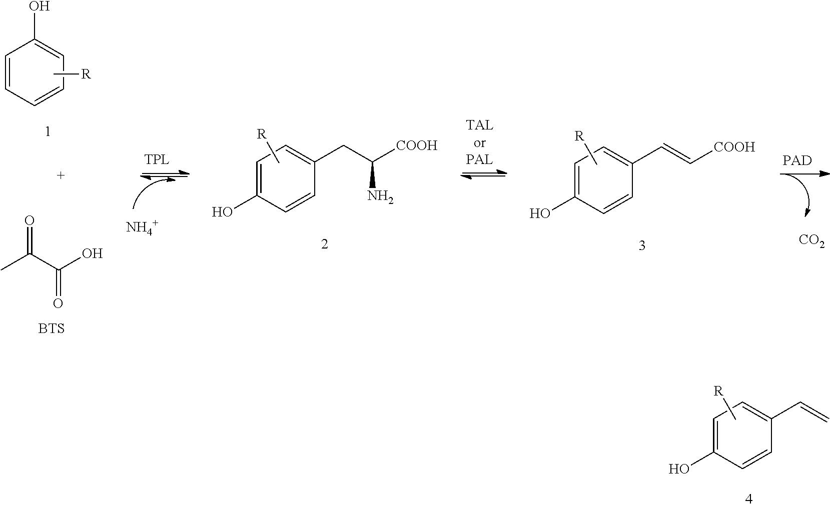

wherein
a) in a manner known per se, optionally substituted phenol 1 is bound to pyruvic acid (BTS) to form optionally substituted tyrosine 2 by means of the catalytic action of a tyrosine phenol-lyase (TPL) and in the presence of ammonium ions,
b) in a manner known per se, ammonia is eliminated from tyrosine 2 by means of the catalytic action of a tyrosine ammonia-lyase (TAL) or a phenyl ammonia-lyase (PAL), in order to produce optionally substituted p-coumaric acid 3, and
c) in a manner known per se, p-coumaric acid 3 is subjected to a decarboxylation reaction by means of the catalytic action of a phenolic acid decarboxylase (PAD), in order to produce the desired, optionally substituted p-vinyl phenol 4;

d) wherein the generated CO₂ is removed from the reaction system, in order to shift the chemical equilibrium of all three reaction steps towards the product side.

The invention is based on the combination of three single reaction steps known per se and on the surprising findings to the effects that not only all three may be performed in a one-pot reaction substantially at the same time without inhibiting each other to a significant extent, but also that conversions of more than 90% are consistently achievable, even using substituted phenols as the starting substrate. This was un-expected, especially since both the educt and all products are formally phenols, so that inhibiting interactions in the three single reaction steps are likely to occur.

By performing the three reaction steps as a one-pot reaction and shifting the chemical equilibrium of the overall reaction by eliminating the carbon dioxide, which is set free in the decarboxylation reaction in step c), from the system, and after optimizing the reaction conditions, the inventors even consistently achieved overall conversions (as calculated over all three steps) of more than 97%, as demonstrated by the exemplary embodiments below. This was not at all foreseeable.

The present invention thus provides a method which enables selective, very fast and almost complete conversion of phenols to p-vinyl phenols of high purity, which was previously not possible by conventional organic-chemical means.

In preferred embodiments, the one-pot reaction according to the invention is performed at a pH of about 8 to 9, more preferably at a pH of about 8, as determined by the inventors by varying the pH value. In this aspect as well, the excellent overall conversions are highly surprising, since the optimum pH of the first two reaction steps is above 10 each, while the decarboxylation reaction is significantly faster and more complete at an acidic to neutral pH than in an alkaline environment, and for the single reaction hardly any conversion was detectable at a pH 9, as again demonstrated by the examples below. This result for the single reaction is consistent with the specialist literature cited at the outset (Rodriguez et al., supra; Jung et al., supra), where the biocatalyzed decarboxylation reaction by means of PAD is performed, for example, at pH 6.5 and 7.0, respectively, although Jung et al. (supra) disclose an optimum pH of 5.8 for the PAD they used.

Performing the inventive one-pot reaction at pH 8 has also proved to be optimal because, at that pH, a non-toxic KPi buffer (potassium phosphate) may be used as an aqueous solvent, whereas at pH 9, more toxic reagents such as sulfonic acid salts (for example, CHES buffer: N-cyclohexyl-2-aminoethanesulfonic acid) would have to be used.

In further preferred embodiments, in step b), a tyrosine ammonia-lyase (TAL) is used as the catalyst, since the inventors detected hardly any conversion with the phenyl ammonia-lyases (PAL) examined. The tyrosine ammonia-lyase (TAL) is preferably used in the form of whole cells containing the recombinant enzyme, since they show sufficiently high activity in the inventive method, thus avoiding isolation of the enzyme from a cell culture, which is complex and may involve high losses.

The above also applies to ferulic acid decarboxylase (FAD), which is preferably used as the catalyst in step c) according to the present invention and is also particularly preferably used in the form of whole cells containing the recombinant enzyme.

In further preferred embodiments, in addition to an aqueous buffer system having an appropriate pH, a water-immiscible co-solvent is used, preferably diethyl ether, more preferably 5% diethyl ether, based on the aqueous buffer system, in order to further increase the overall reaction yield.

The substitution pattern at the starting phenol 1 is not specifically limited, as long as the para-position is unoccupied and unless the substituent(s) interfere(s) with the enzymatic reactions in steps a) to c). As shown in the examples below, the present invention works both with substituents reducing the electron density in the aromatic ring, such as F, Cl, and Br, and with substituents increasing it, such as alkyl or alkoxy. The substituents' size should not exceed a certain level, in order not to hinder coordination with the respective enzyme, particularly in step a), which is why hydrocarbon radicals with no more than 20 carbon atoms are to be preferred, more preferably hydrocarbon radicals with no more than 10 or no more than 6 carbon atoms. The substituent(s) at the ortho- or meta-position of phenol 1 is/are particularly preferably selected from halogens as well as C₁₋₆ alkyl and C₁₋₆ alkoxy residues, such as F, Cl, Br, or (O)CH₃.

### BRIEF DESCRIPTION OF THE DRAWINGS

In the following, the invention is described in more detail with respect to the accomp-anying drawings, wherein:

FIG. 1 shows the conversions in step a) at varying pH values;

FIG. 2 shows the conversions in step b) at varying pH values;

FIG. 3 shows the conversions in step c) at varying pH values;

FIG. 4 shows the overall reaction at varying pH values over time;

FIG. 5 shows the amounts of educt and products at pH 7;

FIG. 6 shows the amounts of educt and products at pH 10;

FIG. 7 shows the overall reaction conversions using varying co-solvents; and

FIG. 8 shows the amounts of educt and products using the optimal reaction conditions.

### EXAMPLES

In the following, exemplary embodiments of the invention are given which are indicated for illustrative purposes only and should not be understood as limiting the scope of the invention.

### General Methods

Chemical reagents were purchased from different commercial sources and used without further purification. Melting points were determined via samples in open capillaries and are uncorrected. ¹H and ¹³C NMR spectra were recorded using a Bruker spectrometer (¹H: 300.13 MHz; ¹³C: 75.5 MHz). Chemical shifts are given in ppm and the coupling constants in Hertz (Hz). Conversions of the aromatic substrates were determined by HPLC on a Shimadzu chromatograph using a Luna C18 column (ø 25 cm×4.6 mm) and a UV detector at varying wavelengths, with conversion variations being about 2% within the systematic error margins. All results of all Synthetic Examples, Reference Examples and examples of the invention are mean values of triplicate reactions.

### Sample Preparation:

A MeCN/H₂O solution (1 mL, 1:1) including 0.1% TFA was added to an aliquot of the reaction mixture (1 mL). The protein was removed by centrifugation and the solution was filtered through a VIVAspin polyether sulfone membrane filter. The resulting solution was analyzed via HPLC under the conditions given below. The relative amounts of the compounds were calculated from the respective peak areas. Column: Luna C18, 5 μm; flow rate: 1 mL/min; temperature: 30° C.; gradient: from 100% H₂O (0.1% TFA) to 100% MeCN (0.1% TFA) within 22 min. Wavelength: 280 nm.

### Synthetic Examples 1 to 3: Production of the Enzyme Preparations

### Synthetic Example 1

Preparation of the TPL as a Cell-Free Extract of Citrobacter freundii M379V

E. coli clones containing the M379V TPL plasmid were grown in LB media that had been prepared by sterilizing a solution (1 L) of the following components in five 1 L Erlenmeyer flasks: tryptone (10 g/L), NaCl (5 g/L), and yeast extract (5 g/L). A pre-culture was prepared by inoculating 100 mL LB media containing ampicillin (100 mg/L). The pre-culture was shaken overnight at 120 rpm and 37° C. Then, the flasks containing ampicillin (100 mg/L) were inoculated with the pre-culture, yielding an initial OD₆₀₀ of 0.05. Subsequently, the cultures were shaken at 120 rpm and 30° C. until an OD₆₀₀ of 0.4 to 0.6 was reached. Protein expression was induced using IPTG (0.5 mM final concentration), and cultures were shaken for 2 h at 20° C. and 120 rpm. Finally, the cells were harvested by centrifugation (8000 rpm, 20 min), washed with potassium phosphate buffer (10 mM, pH 7), resuspended in KPi buffer (50 mM, 180 mM NH₄Cl, 0.04 mM PLP, pH 8) and disrupted by electrosonication (40% amplitude, 1 s pulse on, 2 s pulse off, 5 min). The mixture was harvested by centrifugation (15000 rpm, 15 min) and the supernatant was shock frozen in liquid nitrogen and lyophilized. The lyophilized cell-free extract was stored at 4° C. and used as such in the reactions.

### Synthetic Example 2

Preparation of the TAL of Rhodobacter sphaeroides as a Whole-Cell Catalyst

E. coli clones containing the TAL plasmid were grown in LB media that had been prepared by sterilizing a solution (1 L) of the following components in five 1 L Erlenmeyer flasks: tryptone (10 g/L), NaCl (5 g/L), and yeast extract (5 g/L). A pre-culture was prepared by inoculating 100 mL LB media containing kanamycin (50 mg/L). The pre-culture was shaken overnight at 120 rpm and 37° C. Then, the flasks containing kanamycin (50 mg/L) were inoculated with the pre-culture, yielding an initial OD₆₀₀ of 0.05. Subsequently, the cultures were shaken at 120 rpm and 37° C. until an OD₆₀₀ of 0.5 to 0.7 was reached. Protein expression was induced using IPTG (0.5 mM final concentration), and cultures were shaken for 24 h at 20° C. and 120 rpm. Finally, the cells were harvested by centrifugation (8000 rpm, 20 min), washed with potassium phosphate buffer (10 mM, pH 8), shock frozen in liquid nitrogen and lyophilized. The lyophilized cells were stored at 4° C. and used as such in the reactions.

### Synthetic Example 3

Production of the FAD of Enterobacter sp. as an E. coli Whole-Cell Catalyst

E. coli clones containing the FAD plasmid were grown in LB media that had been prepared by sterilizing a solution (1 L) of the following components in five 1 L Erlenmeyer flasks: tryptone (10 g/L), NaCl (5 g/L), and yeast extract (5 g/L). A pre-culture was prepared by inoculating 100 mL LB media containing kanamycin (50 mg/L). The pre-culture was shaken overnight at 120 rpm and 37° C. Then, the flasks containing kanamycin (50 mg/L) were inoculated with the pre-culture, yielding an initial OD₆₀₀ of 0.05. Subsequently, the cultures were shaken at 120 rpm and 37° C. until an OD₆₀₀ of 0.5 to 0.7 was reached. Protein expression was induced with IPTG (0.5 mM final concentration), and cultures were shaken for 24 h at 20° C. and 120 rpm. Finally, the cells were harvested by centrifugation (8000 rpm, 20 min), washed with potassium phosphate buffer (10 mM, pH 8), shock frozen in liquid nitrogen and lyophilized. The lyophilized cells were stored at 4° C. and used as such in the reactions.

### Reference Examples 1 to 3: Activity Measurements

### Reference Example 1

### Activity of the TPL

For better comparability of enzyme activity, activity of the TPL in the C—C coupling between phenol 1, pyruvic acid or pyruvate, respectively, and ammonia according to step a) was determined by measuring the initial reaction rate by HPLC. One unit of activity was defined as the amount of catalyst that catalyzed the formation of 1 μmol tyrosine per minute under the following conditions: 30° C., KPi buffer 50 mM, pH 8, 850 rpm. The test mixture contained 2-chlorophenol (1b, 23 mM), pyruvate (46 mM), NH₄Cl (180 mM), and the freeze-dried cell-free extract from Synthetic Example 1 that contained the overexpressed TPL (2 mg). Reactions were initiated by adding the enzyme, and the conversion was determined between 1 and 10 min. All measurements were performed at least in triplicate. The ascertained reaction activities corresponded to 0.30 U per mg cell-free extract.

### Reference Example 2

### Activity of the TAL

For better comparability of enzyme activity, activity of the TAL in the conversion of the tyrosine 2 to the cinnamic acid derivate 3 according to step b) was determined by measuring the initial reaction rate by HPLC. One unit of activity was defined as the amount of catalyst that catalyzed formation of 1 μmol cinnamic acid 3 per minute under the following conditions: 30° C., KPi buffer 50 mM, pH 8, 850 rpm. The test mixture contained L-3-chlorotyrosine (2b, 5 mM), NH₄Cl (180 mM), and E. coli whole cells from Synthetic Example 2 overexpressing the TAL (5 mg). Reactions were initiated by adding the enzyme, and the conversion was determined between 5 and 20 min. All measurements were performed at least in triplicate. The ascertained reaction activities corresponded to 9.2 mU per mg of E. coli whole cells.

### Reference Example 3

### Activity of the FAD

For better comparability of enzyme activity, activity of the FAD in the decarboxylation reaction of the cinnamic acid derivative 3 to the vinyl phenol 4 according to step c) was determined by measuring the initial reaction rate by HPLC. One unit of activity was defined as the amount of catalyst that catalyzed formation of 1 μmol vinyl phenol 4 per minute under the following conditions: 30° C., KPi buffer 50 mM, pH 8, 850 rpm. The test mixture contained 3-chloro-4-hydroxycinnamic acid (3-chlorocoumaric acid, 3b, 5 mM), NH₄Cl (180 mM), and E. coli whole cells from Synthetic Example 3 overexpressing the FAD (0.1 mg). Reactions were initiated by adding the enzyme, and the conversion was determined between 1 and 5 min. All measurements were performed at least in triplicate. The ascertained reaction activities corresponded to 2.1 U per mg of E. coli whole cells.

### Reference Examples 4 to 6: Determination of the Optimum pH

### Reference Example 4

### Optimum pH of the TPL

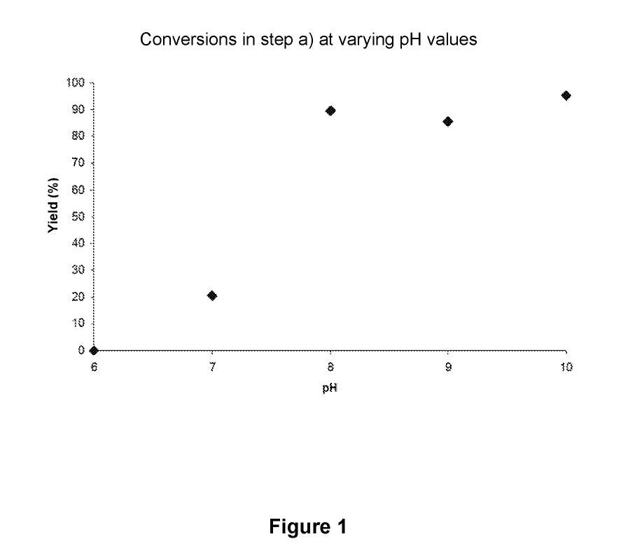

The TPL-catalyzed enzymatic reaction of Reference Example 1 between 2-chlorophenol 1b (23 mM), pyruvate (46 mM), and NH₄⁺ (180 mM) using the freeze-dried cell-free extract from Synthetic Example 1 was repeated with the addition of 0.4 mM pyridoxal phosphate (PLP) as a coenzyme at varying pH values and allowed to proceed for 22 hours. FIG. 1 shows the results for pH values from 6 to 10, from which can be seen that the best conversions to the L-3-chlorotyrosine 2b are obtained at a highly alkaline pH, since, in this range, uncharged ammonia may take part in the reaction. Interestingly, the enzyme remains stable even in a highly alkaline pH range, especially since the best conversion was obtained at pH 10, and thus the optimum is presumed to be even above that value.

### Reference Example 5

### Optimum pH of the TAL

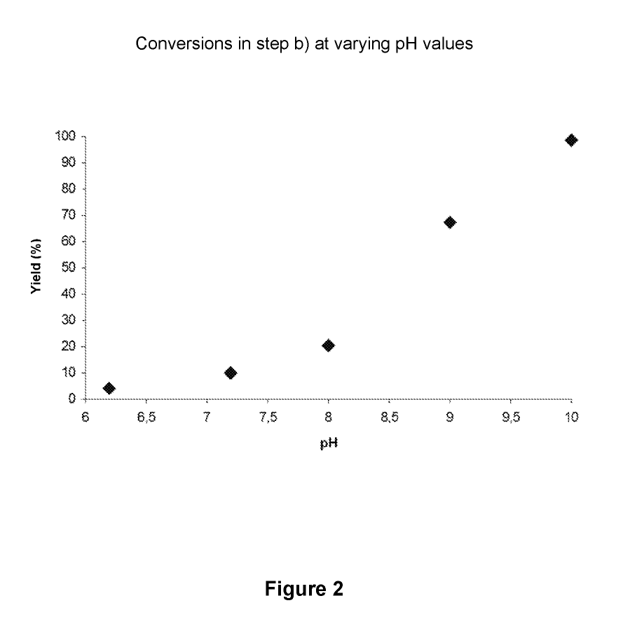

The TAL-catalyzed enzymatic reaction of Reference Example 2 between 3-chlorotyrosine 2b (10 mM) and NH₄⁺ (180 mM) using the whole-cell preparation from Synthetic Example 2 was also repeated with the addition of 0.4 mM pyridoxal phosphate (PLP) as a coenzyme at varying pH values and allowed to proceed for 6 hours. FIG. 2 again shows the results for pH values from 6 to 10, from which can be seen once more that the highly alkaline pH range above pH 10 is to be preferred. In this case, the reaction optimum is sure to be above pH 10, as suggested by extrapolation of a theoretical best-fit curve through the measuring points.

### Reference Example 6

### Optimum pH of the FAD

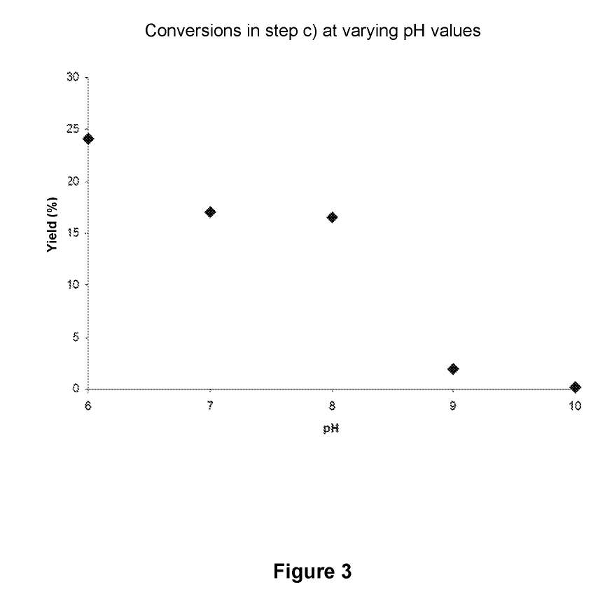

The FAD-catalyzed enzymatic reaction of Reference Example 3, i.e. the decarboxylation reaction of 3-chlorocoumaric acid 3b (10 mM) using the whole-cell preparation from Synthetic Example 3 to the 4-vinyl phenol 4b was repeated with the addition of 0.4 mM pyridoxal phosphate (PLP) as a coenzyme at varying pH values and allowed to proceed for 1 hour. FIG. 3 again shows the results for pH values from 6 to 10, from which can be seen that up to pH 8 conversions comparable to those in the neutral range are achievable, while at pH 9 hardly any conversion occurred and at pH 10 practically no conversion is detectable. In this case, the reaction optimum is sure to be in the acidic pH range, as suggested by an extrapolation of the theoretical best-fit curve and as those skilled in the art would expect for decarboxylation reactions.

### Examples 1 to 4

### One-Pot Reaction of Steps a) to c) at Varying pH Values

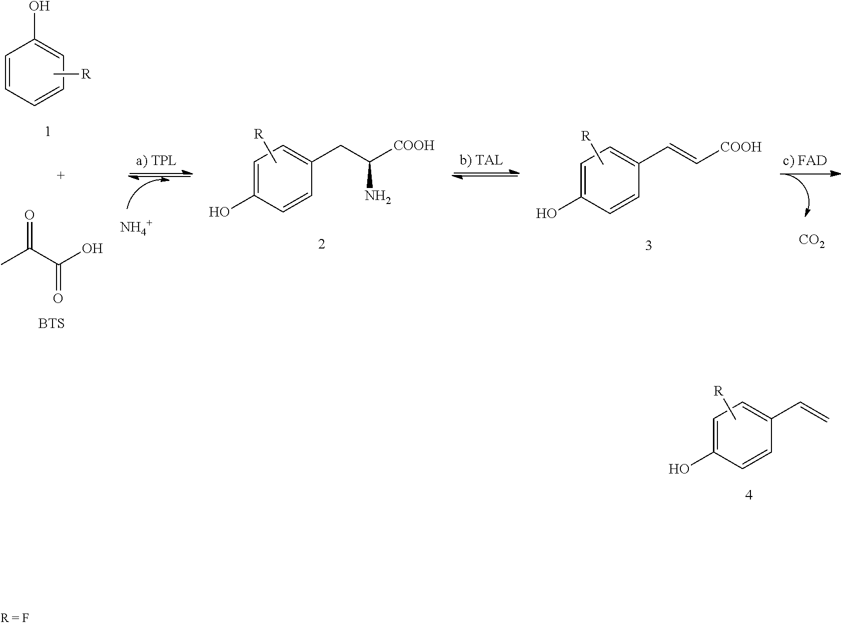

Using the three enzymes of Synthetic Examples 1 to 3, the three-step one-pot reaction was performed using 2-fluorophenol 1a (R=F) under the following conditions: 1a (23 mM); BTS (pyruvate) (46 mM); TPL (4 mg); TAL (20 mg); FAD (5 mg); aqueous buffer (50 mM): KPi for pH 7-8, CHES for pH 9-10; time: 6 h; 20° C.; shaking at 850 rpm. The pH was varied between 7 and 10, and samples were taken at time intervals and analyzed by HPLC for the amount of 2-fluoro-4-vinyl phenol 4a (R=F).

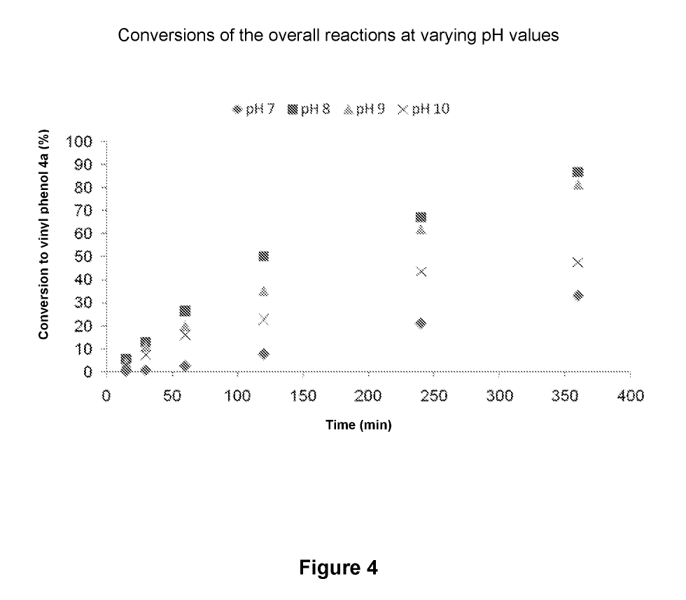

FIG. 4 shows the results of these reactions. It was found that conversions at pH 7 (Example 1; about 30%) and pH 10 (Example 4; about 45%), respectively, were well below those at pH 8 (Example 2; about 85%) and pH 9 (Example 3; about 80%), which, for their part, yielded almost the same conversions, which may be due to low activities of two of the enzymes, TPL and TAL, at pH 7 (see FIGS. 1 and 2) on the one hand, and virtual ineffectiveness of the FAD at pH 10 (see FIG. 3) on the other. Considering the latter, it was surprising that nonetheless significantly better conversions are achievable at pH 10 than at pH 7.

In order to study this phenomenon, Examples 1 and 4 were repeated at pH 7 and pH 10, respectively, and the amounts of educt 1a and of the products 2a to 4a were determined by HPLC at time intervals.

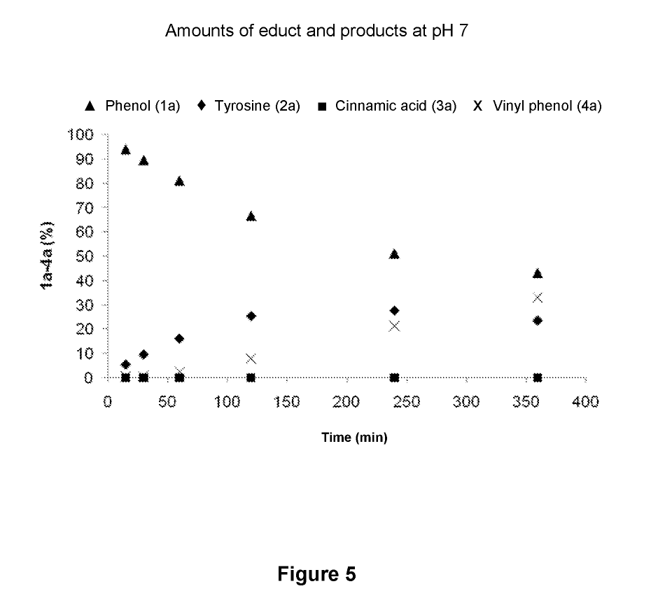

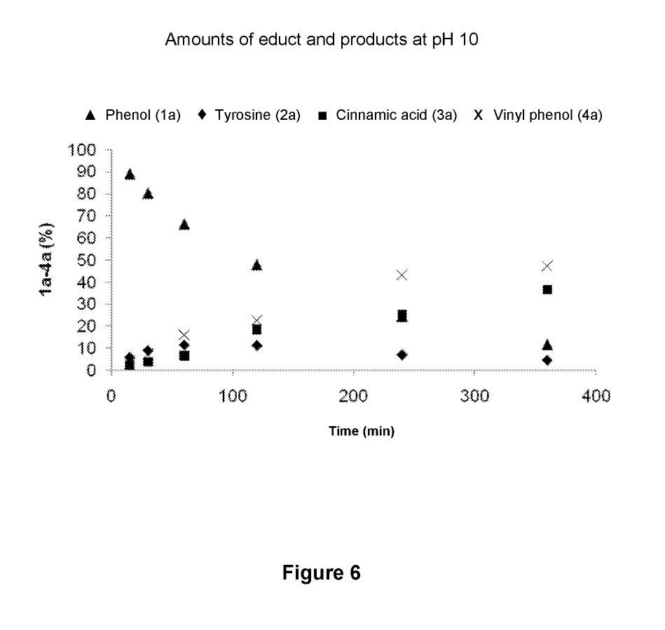

FIGS. 5 and 6 show the results of these experiments for the phenol 1a (▴), the tyrosine 2a (♦), the cinnamic acid 3a (▪), and the desired vinyl phenol 4a (x). This confirmed the above assumptions, as it was found that, at pH 7, the amount of educt 1a decreases only very slowly and more than 40% of the starting phenol is left even after 6 h, all the while practically no cinnamic acid being contained in the reaction mixture. Thus, at pH 7, step a) is the rate-determining partial reaction, whereas the decarboxylation reaction of step c) proceeds the fastest. At pH 10, however, the C—C coupling of step a) is significantly faster, such that, after 6 h, the starting product 1a had decreased to 10% of the initial amount. At the same time, however, the cinnamic acid 3a continuously accumulates in the reaction mixture, which means that, at pH 10, the decarboxylation reaction is the slowest and thus the rate-determining partial reaction.

Due to the necessity of having to use CHES or other, significantly more toxic aqueous buffers than the KPi buffer for pH 8 in order to adjust pH 9, the inventors considered pH 8 as the optimum pH for carrying out the inventive method, despite the conversions being approximately the same.

### Examples 5 to 8

### One-Pot Reaction at pH 8 Using a Co-Solvent

The above Example 2 at pH 8 was thus repeated admixing varying amounts of co-solvents and determining the amounts of the end product, p-vinyl phenol 4a. In Example 5, Example 2 was repeated for comparison purposes without any co-solvent, whereas in Example 6, DMSO was added as an example of a water-miscible solvent (5% (v/v)), in Example 7, diethyl ether was added as an example of a water-immiscible solvent (5% (v/v)), and in Example 8, double the amount of diethyl ether (10% (v/v)) was added, each based on the volume of the aqueous buffer solution.

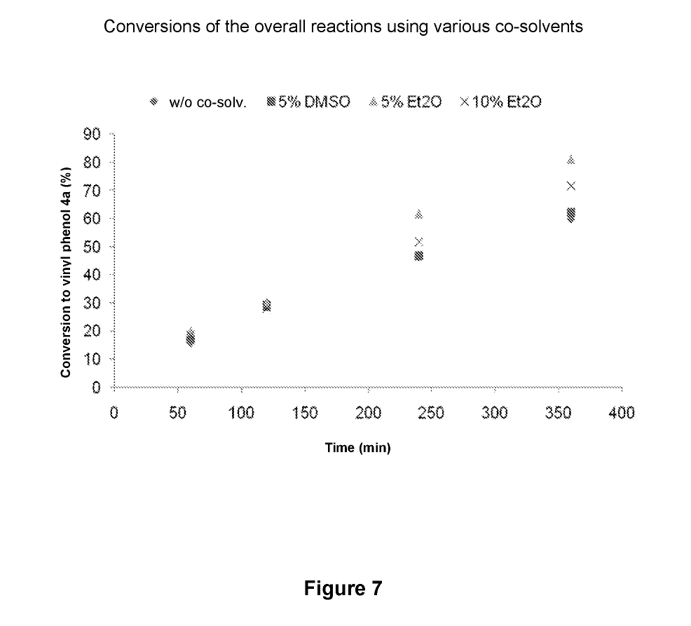

FIG. 7 shows the results of these experiments. As can be seen, the presence of water-miscible DMSO made almost no difference for the conversion to the product 4a, whereas the water-immiscible diethyl ether caused, in an amount of 10% (v/v), a conversion increase of more than 10 percent, but in an amount of 5% (v/v) even more than 20% (v/v), after a reaction time of 6 h.

Without wishing to be bound to any particular theory, the inventors assume that the increasing conversions are due to the formation of two phases, with certain portions of the four different phenols being dissolved in the ether phase; presumably, due to their lower polarity and hydrophilicity, mainly portions of the educt 1a and of the end product 4a. This suppresses potential inhibition of the single reactions, presumably of steps b) and c), and/or shifts the chemical equilibrium of the overall reaction even further to the right. Suppression of inhibiting effects is supported by the fact that no considerable conversion increase is achievable using double the amount of ether, apparently due to excessive amounts of the phenols, possibly of the educt, being dissolved in the organic phase, thus decelerating the reaction rate.

### Example 9

### One-Pot Reaction Under Optimum Conditions

Thus having optimized the pH and co-solvent, the experiment of Example 7 was repeated under the following conditions: 1a (23 mM); pyruvate (46 mM); TPL (4 mg); TAL (20 mg); FAD (5 mg); KPi buffer (50 mM), pH 8; 5% (v/v) Et₂O, time: 6 h; 30° C.; shaking at 850 rpm; and the amounts of the educts 1a and of the products 2a to 4a were determined by HPLC at time intervals.

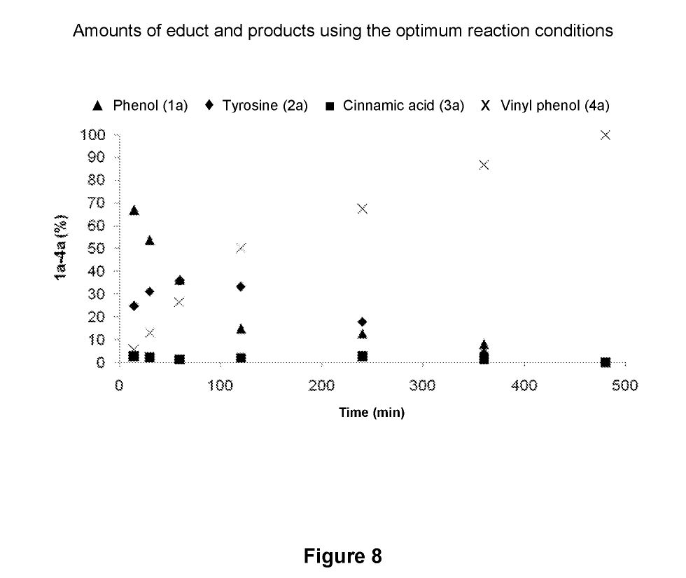

FIG. 8 shows the result of this experiment, the most striking aspects of which being the exceptionally fast decrease of the amount of educt 1a during the first minutes and the conversion to the end product 4a of more than 95% after 6 h.

### Examples 10 to 20

### Preparation of Different Derivatives

Using the optimum reaction conditions from Example 9 or varying them slightly, the substitution pattern (and optionally the amount of the starting substrate, phenol 1, as well) was varied as indicated in Table 1 below, and the conversions, i.e. the relative amounts after terminating the reaction, were determined by HPLC after 24 h and 48 h, respectively, and are also indicated in Table 1.

TABLE 1

| | Phenol | | Conc. 1 | Amount | Amount | Amount | Amount |
|---|---|---|---|---|---|---|---|
| Example | 1 | R | [mM] | 1 [%] | 2 [%] | 3 [%] | 4 [%] |
| 10 | 1a | 2-fluoro | 23 | <1 | <1 | <1 | >97 |
| 11 | 1a | 2-fluoro | 46^{a} | <1 | <1 | <1 | >97 |
| 12 | 1b | 2-chloro | 23 | <1 | <1 | <1 | >97 |
| 13 | 1c | 2-bromo | 23 | <1 | <1 | <1 | >97 |
| 14 | 1d | 2-methyl | 23 | <1 | <1 | <1 | >97 |
| 15 | 1e | 3-fluoro | 23 | <1 | <1 | <1 | >97 |
| 16 | 1e | 3-fluoro | 46^{a,b} | <1 | <1 | <1 | >97 |
| 17 | 1f | 3-chloro | 23 | <1 | <1 | <1 | >97 |
| 18 | 1g | 2,3-difluoro | 23 | <1 | <1 | <1 | >97 |
| 19 | 1g | 2,3-difluoro | 46^{a,b} | <1 | <1 | <1 | >97 |
| 20 | 1h | 2-fluoro-3-chloro | 23^{a} | <1 | <1 | <1 | >97 |

^{a}Double the amount of TAL
^{b}Double the reaction time (48 h)

It was found that irrespective of the type (electron-withdrawing or electron-donating) and the number (one or two) of the substituents R, conversions to the respective product 4 of more than 97% were consistently obtained, and the educts of the three partial reactions were present at less than 1%.

Those skilled in the art will understand that the reaction will proceed with different substitution patterns at starting phenol 1 in a similarly complete manner, unless the substituents significantly interfere with the enzymatic reactions.

### Work-Up:

The reactions were stopped by adding saturated aqueous NH₄Cl solution and extracted with ethyl acetate (3×15 mL). The combined organic phases were dried over Na₂SO₄ and evaporated under reduced pressure. The residue was purified by flash chromatography (20% EtOAc/hexane), and the fractions containing the respective vinyl phenol were combined and evaporated at 100 mmHg, yielding the desired p-vinyl phenols as colourless oils in consistent conversions of 80-90% of the theoretical values. The spectroscopic data of the p-vinyl phenols thus produced are given below.

2-fluoro-4-vinyl phenol 4a:

¹H-NMR (300 MHz, MeOD): δ_{H} [ppm]=5.10 (d, ³J_{2′c,1′}=10.9 Hz, 1H, 2′-H_{c}), 5.59 (dd, ²J_{2′t,2′c}=0.8 Hz, ³J_{2′t,1′}=17.6 Hz, 1H, 2′-Hₜ), 6.59 (dd, ³J_{1′,2′c}=10.9 Hz, ³J_{1′,2′t}=17.6 Hz, 1H, 1′-H), 6.85 (dd, ⁴J_{6,F}=8.8 Hz, ³J_{6,5}=8.5 Hz, 1H, 6-H), 7.02 (dd, ⁴J_{5,3}=2.3 Hz, ³J_{5,6}=8.4 Hz, 1H, 5-H), 7.14 (dd, ⁴J_{3,5}=2.1 Hz, ³J_{3,F}=12.4 Hz, 1H, 3-H).

¹³C-NMR (75 MHz, MeOD): δ_{C} [ppm]=112.3 (C-2′), 114.1 (d, ²J_{3,F}=18.9 Hz, C-3), 118.5 (d, ³J_{6,F}=3.1 Hz, C-6), 123.7 (d, ⁴J_{5,F}=3.1 Hz, C-5), 131.6 (d, ³J_{4,F}=6.2 Hz, C-4), 136.9 (C-1′), 145.9 (d, ²J_{1,F}=13.3 Hz, C-1), 152 (d, ¹J_{2,F}=240.1 Hz, C-2). ¹⁹F (282 MHz, MeOD) δ_{F} [ppm]=−139.7 (dd, ⁴J_{F,6}=9.1 Hz, ⁴J_{F,3}=12.5 Hz).

2-chloro-4-vinyl phenol 4b:

¹H-NMR (300 MHz, MeOD): δ_{H} [ppm]=5.06 (dd, ²J_{2′c,2′t}=0.7 Hz, ³J_{2′c,1′}=10.9 Hz, 1H, 2′-H_{c}), 5.56 (dd, ²J_{2′t,2′c}=0.8 Hz, ³J_{2′t,1′}=17.6 Hz, 1H, 2′-Hₜ), 6.55 (dd, ³J_{1′,2′c}=10.9 Hz, ³J_{1′,2′t}=17.6 Hz, 1H, 1′-H), 6.81 (d, ³J_{6,5}=8.4 Hz, 1H, 6-H), 7.15 (dd, ⁴J_{5,3}=2.2 Hz, ³J_{5,6}=8.4 Hz, 1H, 5-H), 7.32 (d, ⁴J_{3,5}=2.2 Hz, 1H, 3-H).

¹³C-NMR (75 MHz, MeOD): δ_{C} [ppm]=112.4 (C-2′), 117.5 (C-6), 121.8 (C-2), 126.8 (C-3), 128.6 (C-5), 132.0 (C-4), 136.7 (C-1′), 154 (C-1).

2-bromo-4-vinyl phenol 4c:

¹H-NMR (300 MHz, MeOD): δ_{H} [ppm]=5.07 (dd, ²J_{2′c,2′t}=0.6 Hz, ³J_{2′c,1′}=11.0 Hz, 1H, 2′-H_{c}), 5.57 (dd, ²J_{2′t,2′c}=0.6 Hz, ³J_{2′t,1′}=17.6 Hz, 1H, 2′-Hₜ), 6.55 (dd, ³J_{1′,2′c}=10.9 Hz, ³J_{1′,2′t}=17.6 Hz, 1H, 1′-H), 6.84 (d, ³J_{6,5}=8.4 Hz, 1H, 6-H), 7.21 (dd, ⁴J_{5,3}=2.1 Hz, ³J_{5,6}=8.4 Hz, 1H, 5-H), 7.51 (d, ⁴J_{3,5}=2.1 Hz, 1H, 3-H).

¹³C-NMR (75 MHz, MeOD): δ_{C} [ppm]=110.9 (C-2), 112.4 (C-2′). 117.1 (C-6), 127.4 (C-5), 131.7 (C-3), 132.3 (C-4), 136.4 (C-1′), 154.9 (C-1).

2-methyl-4-vinyl phenol 4d:

¹H-NMR (300 MHz, MeOD): δ_{H} [ppm]=2.17 (s, 3H, CH₃), 4.99 (dd, ²J_{2′c,2′t}=1.1 Hz, ³J_{2′c,1′}=10.9 Hz, 1H, 2′-H_{c}), 5.53 (dd, ²J_{2′t,2′c}=1.1 Hz, ³J_{2′t,1′}=17.7 Hz, 1H, 2′-Hₜ), 6.58 (dd, ³J_{1′,2′c}=10.9 Hz, ³J_{1′,2′t}=17.6 Hz, 1H, 1′-H), 6.68 (d, ³J_{6,5}=8.2 Hz, 1H, 6-H), 7.06 (dd, ⁴J_{5,3}=2.2 Hz, ³J_{5,6}=8.2 Hz, 1H, 5-H), 7.13 (d, ⁴J_{3,5}=2.1 Hz, 1H, 3-H).

¹³C-NMR (75 MHz, MeOD): δ_{C} [ppm]=16.2 (CH₃ an C-2), 110.4 (C-2), 115.5 (C-6), 125.5 (C-2), 125.8 (C-5), 129.6 (C-3), 130.6 (C-4), 138.1 (C-1′), 156.5 (C-1).

3-fluoro-4-vinyl phenol 4e:

¹H-NMR (300 MHz, MeOD): δ_{H} [ppm]=5.16 (dd, ²J_{2′c,2′t}=1.4 Hz, ³J_{2′c,1′}=1.3 Hz, 1H, 2′-H_{c}), 5.64 (dd, ²J_{2′t,2′c}=1.3 Hz, ³J_{2′t,1′}=17.8 Hz, 1H, 2′-Hₜ), 6.47 (dd, ⁴J_{2,6}=2.4 Hz, ³J_{2,F}=12.5 Hz, 1H, 2-H), 6.57 (dd, ⁴J_{6,2}=2.5 Hz, ³J_{6,5}=8.6 Hz, 1H, 6-H), 6.74 (dd, ³J_{1′,2′t}=11.3 Hz, ³J_{1′,2′t}=17.8 Hz, 1H, 1′-H) 7.35 (dd, ³J_{5,6}=8.7 Hz, ⁴J_{5,F}=8.8 Hz, 1H, 5-H).

¹³C-NMR (75 MHz, MeOD): δ_{C} [ppm]=103.5 (d, ²J_{2,F}=25.0 Hz, C-2), 112.6 (d, ⁴J_{2′,F}=2.7 Hz, C-2′), 113.4 (d, ⁴J_{6,F}=4.8 Hz, C-6), 117.8 (d, ³J_{4,F}=12.6 Hz, C-4), 128.8 (d, ³J_{1′,F}=5.8 Hz, C-1), 130.3 (d, ³J_{5,F}=3.5 Hz, C-5), 159.9 (d, ²J_{2,F}=11.9 Hz, C-1), 162.3 (d, ¹J_{3,F}=247.4 Hz, C-3); ¹⁹F (282 MHz, MeOD): δ_{F} [ppm]=−119.8 (dd, ⁴J_{F,5}=8.8 Hz, ³J_{F,2}=12.6 Hz).

3-chloro-4-vinyl phenol 4f:

¹H-NMR (300 MHz, MeOD): δ_{H} [ppm]=5.18 (dd, ²J_{2′c,2′t}=1.2 Hz, ³J_{2′c,1′}=11.0 Hz, 1H, 2′-H_{c}), 5.60 (dd, ²J_{2′t,2′c}=1.2 Hz, ³J_{2′t,1′}=17.5 Hz, 1H, 2′-Hₜ), 6.71 (dd, ⁴J_{6,2}=2.5 Hz, ³J_{6,5}=8.6 Hz, 1H, 6-H), 6.79 (d, ³J_{2,6}=2.5 Hz, 1H, 2-H), 6.98 (dd, ³J_{1′,2′c}=11.0 Hz, ³J_{1′,2′t}=17.6 Hz, 1H, 1′-H), 7.47 (d, ³J_{5,6}=8.6 Hz, 1H, 5-H).

¹³C-NMR (75 MHz, MeOD): δ_{C} [ppm]=113.8 (C-2′), 115.7 (C-6), 116.8 (C-2), 128.1 (C-4), 128.3 (C-5), 133.7 (C-1′), 134.4 (C-3), 159.2 (C-1).

2,3-difluor-4-vinyl phenol 4g:

¹H-NMR (300 MHz, MeOD): δ_{H} [ppm]=5.25 (dd, ²J_{2′c,2′t}=1.1 Hz, ³J_{2′c,1′}=11.4 Hz, 1H, 2′-H_{c}), 5.70 (dd, ²J_{2′t,2′c}=1.1 Hz, ³J_{2′t,1′}=17.8 Hz, 1H, 2′-Hₜ), 6.68 (ddd, ⁵J_{6,3F}=2.0 Hz, ⁴J_{6,2F}=8.0 Hz, ³J_{6,5}=8.5 Hz, 1H, 6-H), 6.72 (dd, ³J_{1′,2′c}=11.3 Hz, ³J_{1′,2′t}=17.8 Hz, 1H, 1′-H), 7.11 (ddd, 5J_{5,2F}=2.3 Hz, ⁴J_{5,3F}=8.3 Hz, ³J_{5,6}=8.5 Hz, 1H, 5-H).

¹³C-NMR (75 MHz, MeOD): δ_{C} [ppm]=113.7 (dd, ³J_{6,2F}=3.2 Hz, ⁴J_{6,3F}=3.2 Hz, C-6), 115.1 (d, ⁴J_{2′,3F}=5.1 Hz, C-2′), 119.2 (dd, ³J_{4,2F}=1.1 Hz, ²J_{4,3F}=9.7 Hz, C-4), 122.2 (dd, ³J_{5,3F}=4.5 Hz, ⁴J_{5,2F}=4.5 Hz, C-5), 129.6 (dd, ⁴J_{1′,2F}=3.1 Hz, ³J_{1′,3F}=3.3 Hz, C-1′), 141.8 (dd, ²J_{2,3F}=14.5 Hz, ¹J_{2,2F}=241.2 Hz, C-2), 147.5 (dd, ³J_{1,3F}=2.8 Hz, ²J_{1,2F}=10.2 Hz, C-1), 150.7 (dd, ²J_{3,2F}=10.9 Hz, ¹J_{3,3F}=248.4 Hz, C-3);

¹⁹F (282 MHz, MeOD): δ_{F} [ppm]=−146.1 (ddd, ⁵J_{3F,6}=2.0 Hz, ⁴J_{3F,5}=8.1 Hz, ³J_{3F,2F}=18.4 Hz, 1F, F an C-3), −165.1 (ddd, ⁵J_{2F,5}=2.4H, ⁴J_{2F,6}=8.0 Hz, ³J_{2F,3F}=18.3 Hz, 1F, F an C-2).

3-chloro-2-fluoro-4-vinyl phenol 4h:

¹H-NMR (300 MHz, MeOD): δ_{H} [ppm]=5.27 (d, ³J_{2′c,1′}=11.1 Hz, 1H, 2′-H_{c}), 5.68 (dd, ²J_{2′t,2′c}=0.9 Hz, ³J_{2′t,1′}=17.5 Hz, 1H, 2′-Hₜ), 6.85 (dd, ⁴J_{6,F}=8.6 Hz, ³J_{6,5}=8.6 Hz, 1H, 6-H), 6.96 (dd, =11.0 Hz, ³J_{1′,2′t}=17.5 Hz, 1H, 1′-H), 7.29 (dd, ⁵J_{5,F}=2.0 Hz, ³J_{5,6}=8.7 Hz, 1H, 5-H).

¹³C-NMR (75 MHz, MeOD): δ_{C} [ppm]=115.4 (C-2′), 117.1 (d, ³J_{6,F}=3.1 Hz, C-6), 121.5 (d, ²J_{3,F}=15.1 Hz, C-3), 122.2 (d, ⁴J_{5,F}=4.0 Hz, C-5), 129.2 (d, ³J_{4,F}=1.3 Hz, C-4), 132.9 (d, ⁴J_{1′,F}=3.1 Hz, C-1′), 146.8 (d, ²J_{1,F}=13.3 Hz, C-1), 148.9 (d, ¹J_{2,F}=241.2 Hz, C-2); ¹⁹F (282 MHz, MeOD): δ_{F} [ppm]=−139.6 (dd, ⁴J_{F,6}=8.8 Hz, ⁵J_{F,5}=2.0 Hz).

The present invention thus provides a one-pot method for preparing p-vinyl phenols, according to which a multitude of compounds may be prepared in an efficient and inexpensive manner, which was nowhere near possible according to the state of the art.

1.-10. (canceled)

11. A biocatalytic method for preparing p-vinyl phenols, the method comprising a three-step, one-pot reaction according to a following reaction scheme: 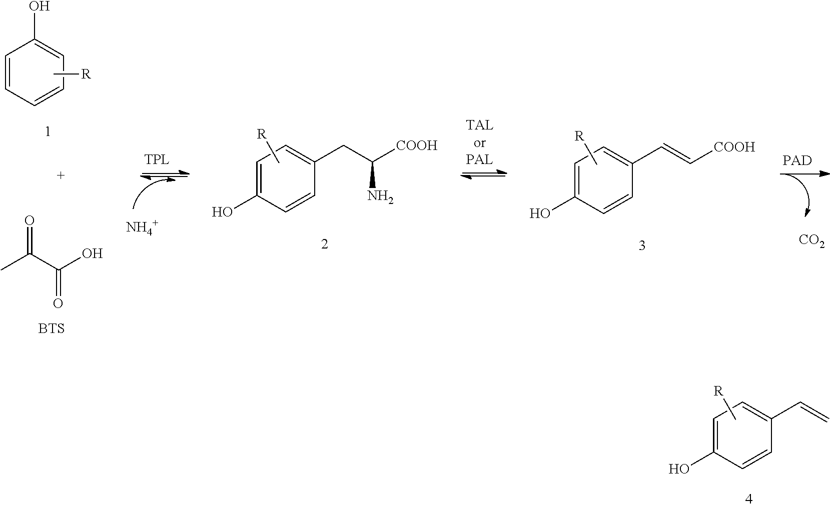
wherein R is an optional substituent and wherein the reaction steps comprise:
(a) binding optionally substituted phenol (1) to pyruvic acid (BTS) to form optionally substituted tyrosine (2) by catalytic action of a tyrosine phenol-lyase (TPL) and in a presence of ammonium ions,
(b) eliminating ammonia from tyrosine (2) by catalytic action of a tyrosine ammonia-lyase (TAL) or a phenyl ammonia-lyase (PAL) to produce optionally substituted p-coumaric acid (3), and
(c) subjecting p-coumaric acid (3) to a decarboxylation reaction by catalytic action of a phenolic acid decarboxylase (PAD) to produce an optionally substituted p-vinyl phenol (4);
and further comprising removing CO₂ generated from the reaction steps to shift chemical equilibrium of the reaction steps toward a product side.

12. The method according to claim 11, wherein the reaction is performed at a pH of about 8 to 9.

13. The method according to claim 12, wherein the reaction is performed at a pH of about 8.

14. The method according to claim 11, wherein in step (b) the catalytic action uses a catalyst comprising a tyrosine ammonia-lyase (TAL).

15. The method according to claim 14, wherein the tyrosine ammonia-lyase (TAL) is used in a form of whole cells containing recombinant enzyme.

16. The method according to claim 11, wherein in step (c) the catalytic action uses a catalyst comprising a ferulic acid decarboxylase (FAD).

17. The method according to claim 16, wherein the ferulic acid decarboxylase (FAD) is used in a form of whole cells containing recombinant enzyme.

18. The method according to claim 11, wherein the one-pot reaction is performed in an aqueous buffer system in a presence of a water-immiscible co-solvent.

19. The method according to claim 18, wherein the co-solvent comprises diethyl ether.

20. The method according to claim 19, wherein the diethyl ether is present in an amount of 5% (v/v) based on the aqueous buffer system.

21. The method according to claim 11, wherein the phenol (1) is substituted in at least one of ortho- and meta-positions with at least one substituent R selected from halogens, C₁₋₆ alkyl, and C₁₋₆ alkoxy.
